(19) United States
(12) Patent Application Publication
(10) Pub. No.: US 2017/0332947 A1
(43) Pub. Date: Nov. 23, 2017

(54) SYSTEM AND METHODS FOR DIPLOPIA ASSESSMENT

(71) Applicant: Nicholas Olof SUNDIN, El Paso, TX (US)

(72) Inventor: Nicholas Olof SUNDIN, El Paso, TX (US)

(21) Appl. No.: 15/524,309

(22) PCT Filed: Nov. 4, 2015
(86) PCT No.: PCT/US2015/058981

§ 371 Date: May 4, 2017

Related U.S. Application Data
(60) Provisional application No. 62/077,233, filed on Nov. 8, 2014.

(51) Int. Cl. A61B 5/11; A61B 3/08; A61B 5/03; A61B 5/00; A61B 3/00

(52) U.S. Cl. CPC A61B 5/1114; A61B 3/08; A61B 5/032; A61B 3/00; A61B 5/00

(57) ABSTRACT

Embodiments of the present disclosure comprise systems and methods for assessing diplopia. Particular embodiments include a computer processor and a headset comprising a visual display and a sensor configured to detect movement of the headset.

### CROSS-REFERENCE TO RELATED APPLICATIONS

This application claims priority to U.S. Provisional Patent Application Ser. No. 62/077,233, filed Nov. 8, 2014, the contents of which are incorporated by reference herein.

### BACKGROUND INFORMATION

### I. Field of the Invention

Embodiments of the present invention relate generally to systems and methods for assessing diplopia in a pair of eyes of a person. Specific embodiments of the present invention relate to an automated system of measuring diplopia utilizing a headset worn by a subject. Diplopia, commonly known as double vision, is a condition in which a person's eyes do not move together. This symptom can be present in conditions such as strabismus as well as in cases of trauma of or near the eye. As explained more fully below, embodiments of the present disclosure can be used to test individuals with this condition. For example, exemplary embodiments can be used for initial diagnosis and to monitor for progression of the disorder during treatment. Embodiments of the present disclosure can be applied inside and outside of traditional healthcare settings.

### II. Background and Description of Related Art

Conventional methods to measure double vision typically include manual methods. For quantitative results, trial prisms that bend light to varying degrees can be placed in front of one of a patient's eyes until they see a single image. This method works best when a patient has the same or similar angle of deviation for their whole visual field. However, this is not always the case with individual experiencing diplopia. In the case of non-uniform degrees of double vision (depending on where a person's eyes are looking they may or may not see double), a more qualitative test will be conducted. An examiner will hold a stimulus in front of the patient and move this stimulus while simultaneously asking whether the person sees a double copy of this stimulus object. This technique takes practice, and results can vary with the skill of the practitioner. Most importantly, this mapping of the field does not produce quantitative data, which could be useful for analysis of the type and basis of the visual dysfunction, documenting tangible improvement after a treatment, or simply storage in a digital medical record.

Automated methods for measuring strabismus do exist, but tools for measuring specialized gaze defects such as double vision are less common. For many hospitals in poorer areas, cost is the main factor hampering the adoption of any of these technologies. The high cost of currently available diagnostic machines is due to the need for special cameras for eye tracking. Finally, these automated technologies usually require the user to press buttons in response to certain stimuli. Such actions can be confusing for patients, especially the elderly or young. This confusion can sometimes lead to inaccurate test results.

It is apparent that a new, more accessible and intuitive system is needed for testing subjects with diplopia and other gaze-related disorders.

### SUMMARY

Particular embodiments of the invention disclosed herein utilize a virtual reality headset to examine and record the extent of double vision in a patient. In specific embodiments, the system contains the following components: first, a head-mounted virtual reality display with one or more attached motion, position, and/or rotation sensors. Second, a computation device, such as a processor found in a computer or mobile device to which the display and sensors are coupled (e.g. either integrally or wirelessly). Software running on the computation device controls the testing process after the operator selects desired options. During the testing process a visual stimulus is displayed to each eye via the display.

Prior to the test, the person can be instructed to move his or her head until they no longer see a double image. The computer will adjust the image in one of their eyes until the stimulus is placed in such a configuration that the person no longer sees a double image. The head-mounted motion sensors, which are a standard part of modern virtual reality systems, allow the computer to precisely track and record all head movements. Analysis of this data stream by embodiments as disclosed herein allows the computer to determine when the person stops trying to align the two images, an indication that the person has found a placement of both images which is perceived as a single image. This head-motion analysis and alignment detection is a significant advance over the prior art, allowing the test to be self-paced, intuitive, and faster at testing than previous devices.

Exemplary embodiments include a system for assessing diplopia. In particular embodiments, the system comprises a headset and a computer processor. In certain embodiments, the headset comprises a sensor configured to detect movement of the headset, and a visual display configured to display a first object and a second object. In specific embodiments, the computer processor is configured to receive an input from the sensor, where the input is correlated to movement of the headset from a first position to a second position. In particular embodiments, the computer processor is configured to transmit an output signal to move the first object or move the second object within the visual display, where the movement of the first or second object is in response to the first input received from the sensor. In certain embodiments, the computer processor is configured to quantify the movement of the headset from the first position to the second position.

In specific embodiments, the first object and the second object are not aligned in the visual display when viewed by a person with diplopia with the headset in the first position, and the first object and the second object are aligned in the visual display when viewed by the person with diplopia with the headset in the second position. In certain embodiments, the sensor is an accelerometer or magnetic sensor. In particular embodiments, the sensor is configured to detect orthogonal position data of the headset. In specific embodiments, the sensor is configured to detect rotational position data of the headset. In certain embodiments, the computer processor is configured to receive the input and transmit the output signal via a wireless transmission. In particular embodiments, the system is not configured to detect eye movement of a person when the person is wearing the headset. Specific embodiments further comprise an audio transmitter configured to provide audible instructions to a person during operation. In certain embodiments, the headset is a virtual reality headset. In certain embodiments, the visual display is configured to cover a field of view of a person wearing the headset.

Exemplary embodiments include a method of assessing diplopia in a person, where the method comprises: (i) displaying a first object and a second object in a visual display of a headset worn by the person; (ii) detecting movement of the head of the person from a first position to a second position; and (iii) moving the first object or the second object in the visual display of the headset in response to the movement of the head of the person. In certain embodiments, wherein the first object and the second object do not appear to the person to be aligned when the head of the person is in the first position; and the first object and the second object do appear to the person to be aligned when the head of the person is in the second position.

In particular embodiments of the method, the movement is detected via a sensor coupled to the headset. In specific embodiments, the sensor is an accelerometer or magnetic sensor. In certain embodiments, the movement detected by the sensor is orthogonal movement. In certain embodiments, the movement detected by the sensor is rotational movement. Particular embodiments comprise transmitting data from the sensor to a computer processor. In specific embodiments, the computer processor records movement of the head of the person from the first position to the second position. In certain embodiments, the movement of the head of the person from the first position to the second position is an indication of diplopia. In particular embodiments, the computer processor: receives an input signal from the sensor correlating to movement of the head of the person; and transmits an output signal to move the first object or the second object in the visual display of the headset.

In specific embodiments of the method, the headset is a virtual reality headset. Certain embodiments further comprise providing instructions to the person to move the head of the person to align the first object and the second object, wherein providing instructions occurs after step (i) and before step (ii). Certain embodiments further comprise repeating steps (i), (ii) and (iii), where the first object and the second object are displayed in different locations in the visual display of the headset worn by the person in each iteration of step (i). In particular embodiments, the method does not comprise detecting eye movement of the person.

Exemplary embodiments include a method of assessing diplopia in a person, where the method comprises: displaying a first object and a second object in a visual display of a headset worn by the person, where the first object and the second object do not appear to the person to be aligned; and where the first object appears to the person to be moving along a first path having a first plurality of locations. In certain embodiments the method also includes recording movement of the head of the person along a second path as the person attempts to align the first object with the second object, where the second path comprises a second plurality of locations. In particular embodiments, the method also includes comparing the first plurality of locations to the second plurality of locations to establish a plurality of deviation angles; and recording the deviation angles of the movement of the head of the person along the second path.

In certain embodiments, the first path of the first object extends across the visual field of the person. In particular embodiments, the movement of the head of the person along the second path is recorded by a computer processor receiving an input signal from a sensor coupled to the headset. In specific embodiments, the sensor is an accelerometer or magnetic sensor. In certain embodiments, the sensor is configured to detect orthogonal position data of the headset. In particular embodiments, the sensor is configured to detect rotational position data of the headset. In specific embodiments, the computer processor is configured to receive the input and transmit the output signal via a wireless transmission. In certain embodiments, the method does not comprise detecting eye movement of the person.

Particular embodiments further comprise providing audible instructions to the person during operation. In specific embodiments, the headset is a virtual reality headset. In certain embodiments, the visual display is configured to cover a field of view of a person wearing the headset.

In the following, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The terms “about”, “approximately” and “substantially” mean, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.” The use of the term “fluid” includes both liquid and gasses.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of this disclosure, are given by way of illustration only, since various changes and modifications within the spirit and scope of the invention will be apparent to those skilled in the art from this detailed description.

### BRIEF DESCRIPTION OF THE FIGURES

FIG. 1 is a schematic view of a system according to an embodiment of the present disclosure.

FIG. 2 is a schematic view of right and left displays of the embodiment of FIG. 1.

FIG. 3 is a schematic view of a combined display of the embodiment of FIG. 1.

FIG. 4 is a schematic view the embodiment of FIG. 1 during operation.

FIG. 5 is a schematic view of right and left displays of the embodiment of FIG. 1 during operation.

FIG. 6 is a schematic view of a combined display of the embodiment of FIG. 1 during operation.

FIG. 7 is a flowchart of a method performed by the embodiment of FIG. 1.

### DETAILED DESCRIPTION OF ILLUSTRATIVE EMBODIMENTS

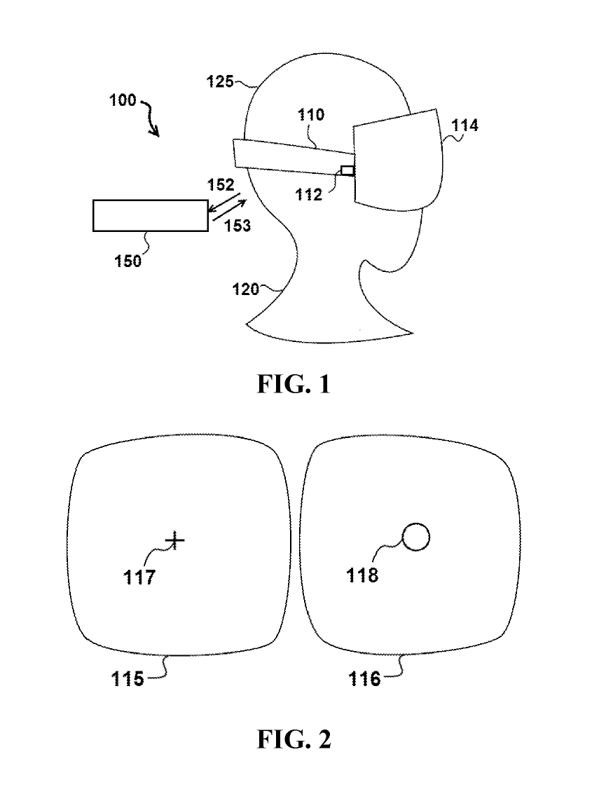

Referring now to FIG. 1, a system 100 for assessing diplopia comprises a headset 110 and a computer processor 150. In the embodiment shown, headset 110 comprises a sensor 112 configured to detect movement of headset 110. During use, sensor 112 can detect movement of headset 110 as well as the movement of a head 125 of person 120 (e.g. if person 120 moves head 125 while wearing headset 110). Headset 110 also comprises a visual display 114.

As shown in FIG. 2, visual display 114 provides a left view 115 visible to the person's left eye and a right view 116 visible to the person's right eye. In the embodiment shown, a first object 117 is shown in left view 115, and a second object 118 is shown in right view 116. Visual display 114 provides a combined view 119 (e.g. as viewed by person 120 in their binocular field of view) that includes first object 117 and second object 118.

System 100 can be used to assess visual disorders (including for example, diplopia) of person 120 in an efficient an intuitive manner for person 120. An overview of the operation of system 100 will be provided initially, followed by a description of more particular aspects. In a particular embodiment, system 100 can be configured so that first object 117 and second object 118 are not aligned in combined view 119 of visual display 114 when viewed by a person 120 with diplopia wearing headset 110. Person 120 can then receive instructions to move his or her head 125 in an effort to align first object 117 and second object 118.

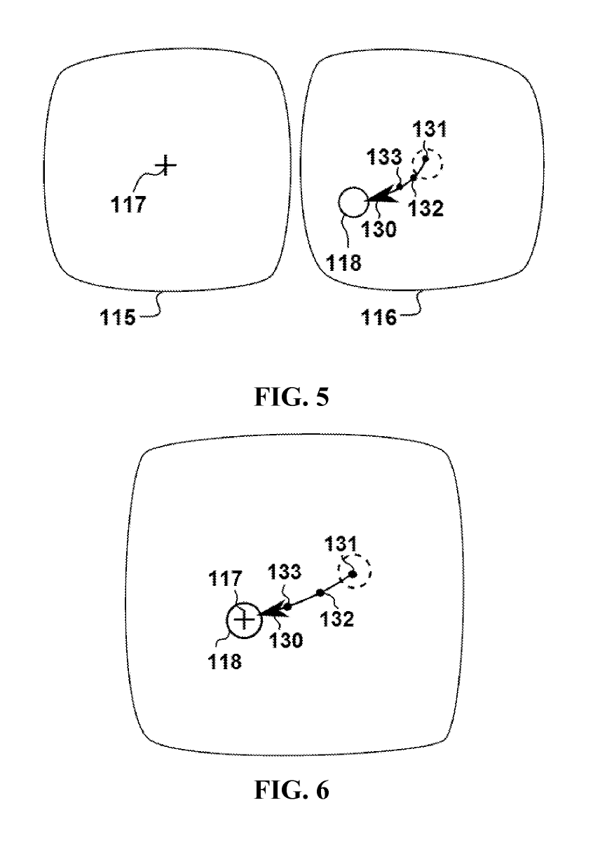

Computer processor 150 can receive an input 152 from sensor 112 that is correlated to the movement of headset 110 and head 125 of person 120. Computer processor 150 can also be configured to move first object 117 and/or second object 118 in response to the movement of headset 110 (e.g. via an output 153 transmitted from computer processor 150 to visual display 114). Accordingly, when person 120 moves his or her head 125 and headset 110 from a first position 121 shown in FIG. 1 to a second position 122 shown in FIG. 4, first object 117 and/or second object 118 are also moved within visual display 114 as shown in FIGS. 5 and 6.

In a specific embodiment, person 120 may move his or her head 125 and headset 110 in an effort to align first object 117 and second object 118. For example, when person 120 initially looks into visual display 114, first and second objects 117 and 118 are not aligned (e.g. as a result of diplopia experienced by person 120). Person 120 can then receive instructions to move his or head 125 in an attempt to align first and second objects 117 and 118. In the embodiment shown, as person 120 moves his or head 125, sensor 112 detects movement of head 125 and headset 110. Computer processor 150 receives input 152 from sensor 112 and transmits output signal 153 to move second object 118 within visual display 114 (as shown in right view 116 and combined view 119). In this embodiment, second object 118 is moved in response to the movement of head 125 until second object 118 and first object 117 are aligned based on instructions provided to person 120. Computer processor 150 can receive data from sensor 112 and quantify the movement of headset 110 from first position 121 to second position 122. The quantification of such movement of headset 110 can be used to assess diplopia in person 120.

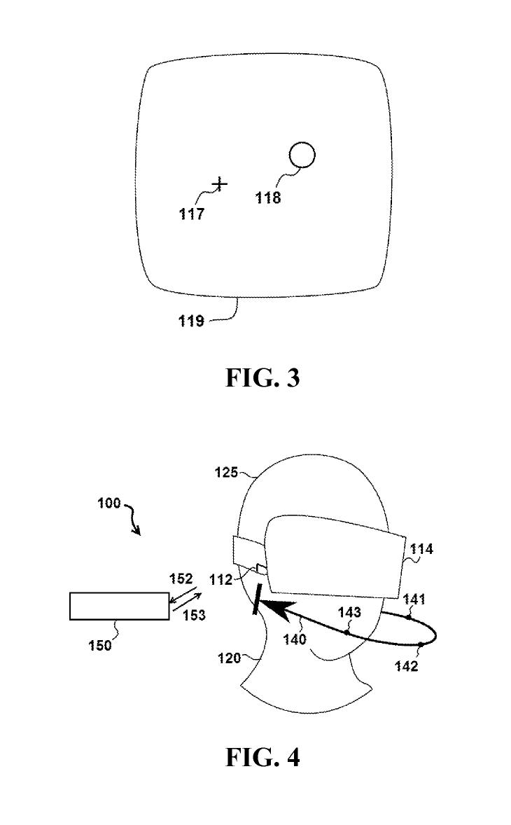

Based on the provided instructions, after person 120 perceives first and second objects 117 and 118 to be aligned, person 120 will not continue to move his or her head 125. Sensor 112 can detect when movement of head 125 has not occurred (or has been below a particular threshold) for a designated period of time when person 120 has stopped moving head 125 in an effort to align first and second objects 117 and 118. When this designated period of time has been met, computer processor 150 can continue or conclude the visual disorder assessment. If the assessment is continued, first and second objects 117 and 118 can be placed in a different portion of visual display 114 than those shown in FIGS. 2 and 3, and the assessment process repeated. In this manner, the diplopia of person 120 can be assessed in different areas of the field of vision of person 120. The assessment can also be continued with first and second objects 117 and 118 placed in the same locations as shown in FIGS. 2 and 3 to confirm the initial results and evaluate the repeatability of the assessment.

In other embodiments, first and second objects 117 and 118 may not appear to be stationary to person 120 during testing. For example, at the initial stages of the assessment person 120 may maintain his or her head 125 in a stationary position as computer processor 150 moves first object 117 or second object 118 within visual display 114 (e.g. along path 130 having a plurality of positions 131-133 shown in FIGS. 5 and 6). Person 120 can then move his or her head 125 along path 140 having a plurality of positions 141-143 as shown in FIG. 4 in an attempt to align first and second objects 117 and 118. Computer processor 150 can compare first plurality of locations 131-133 to second plurality of locations 141-143 to establish a plurality of deviation angles. Computer processor 150 can also record the deviation angles of the movement of head 125 along the path 140. In this manner, deviation angles (e.g. away from normal vision) are continuously recorded to form a map of deviations across the visual field.

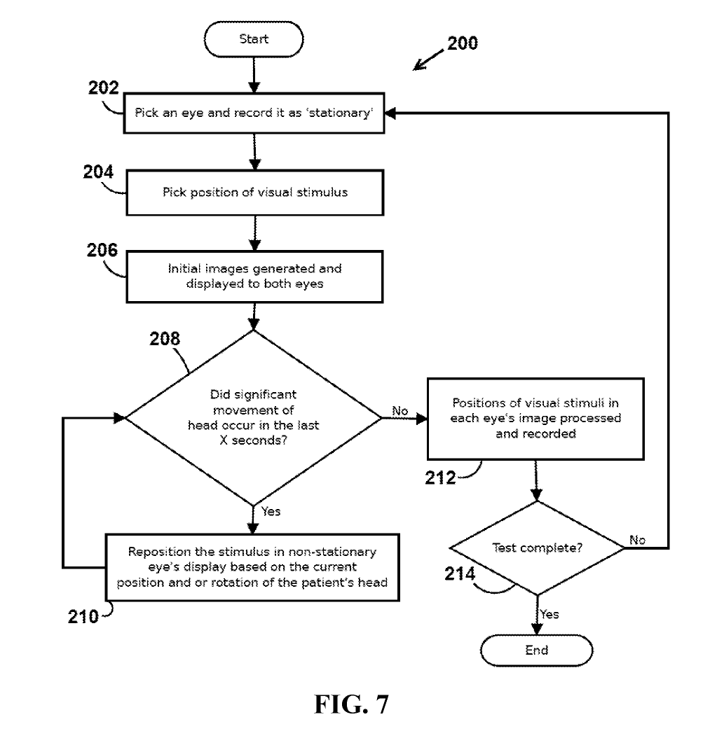

In particular embodiments, computer processor 150 may be configured to perform the steps of method 200 as outlined in FIG. 7. The following description of steps includes reference to components of system 100 shown and described in previous figures that are not illustrated in FIG. 7. In the embodiment shown, computer processor is configured to select either first object 117 in left view 115 or second object 118 in right view 116 as a stationary object in step 202. The object that was not selected as the stationary object can then be designated as the moving object or visual stimulus in step 204. The initial images can be generated and displayed in left and right views 115 and 116 in step 206. In step 208, computer processor 150 can determine if significant (e.g. greater than a pre-determined threshold) movement of head 125 occurred within a specified time frame. If sufficient movement did occur, computer processor 150 can reposition the stimulus (e.g. either first or second object 117, 118 designated as the moving object) within the appropriate view based on the current location of head 125. The location of head 125 can include both orthogonal position (e.g. X-Y-Z coordinate data) and rotational position data of head 125.

In particular embodiments, headset 110 may be configured as a virtual reality device, and in a specific embodiment headset 110 can be an Oculus Rift device. In certain embodiments, computer processor 150 may be integral with headset 110, while in other embodiments computer processor 150 may be separate from headset 110. In embodiments in which computer processor 150 is a separate component, computer processor 150 may communicate with headset 110 via a wireless or wired coupling. In specific embodiments, computer processor 150 may be located in a laptop or desktop computer, or in a mobile device such as a phone.

In particular embodiments, sensor 112 may be an accelerometer, a magnetic sensor or other sensor configured to detect the position, motion and/or rotation of headset 110.

In practice, an assessment of person 120 using system 100 may comprise several aspects. For example, the assessment may begin with an information session telling person 120 specific instructions to follow during the assessment. In addition, the assessment can comprise affixing the headset 110 person 120, followed by one or more assessment cycles. In each cycle, the amount of divergence from normal binocular visual gaze can be measured at a different or identical point on a person's binocular visual field. As previously described, the assessment involves showing the person an image in each of their eyes and detecting if the person perceives a single or double image. If the person sees a double image, the person can be instructed to move one eye's image by moving his or her head so that they see a single image.

Accordingly, embodiments of the present disclosure can measure the amount of deviation (where normal is no deviation) from the normal binocular gaze at a number of points across a subject's binocular field of view. Results from this system can be used directly by doctors or further processed by computer algorithms to extract useful information, such as identifying which weak or damaged eye muscles are affecting vision. One way to visualize divergences from normal vision across a field of view could be with a heat-map showing the degree of angular divergence from normal gaze. Such a device is useful for diagnosing and measuring the extent of and improvement in conditions such as strabismus or trauma of or near the eye. Finally, the intuitive operation by the subject comes from the novel concept of using the person's own head motions to signal the divergence of the eyes

Operational benefits of systems and methods disclosed herein are achieved by the adjustment superimposition of the images by way of head movement and/or rotation and the signaling of the person's ‘satisfaction’ by their lack of further head motion. As used herein, ‘satisfaction’ indicates that the person has adjusted the translation of an image such that both stimuli appear aligned on top of each other, as a single stimulus.

The rules of geometry incorporated into the ideal virtual environment provide that virtual objects at different angles and distances from the subject will appear as single objects to an individual with normal binocular vision. A person with diplopia will have difficulties viewing objects in this virtual environment that correspond directly to their difficulties with a real visual environment. The intuitive use of head motion allows the person to edit the virtual reality display for one eye, such that the visual defect is corrected, at least for the target object.

In practice one eye can be tested initially, and the other eye can then be tested in a similar manner. The geometric difference between the ideal virtual environment and this “corrected” virtual environment provides a quantitative measure of the visual disability relative to each eye. Since the brain has mechanisms that can suppress awareness of visual anomalies, this “correction” protocol is a more sensitive test of the quantitative deviation of an individual's binocular visual function than one based on the reporting of double vision. It should prove especially useful in evaluating trauma to the eye, as there is often a considerable difference in the angles of deviation in different portions of the visual field, and quantitative information is usually not collected or recorded.

All of the devices, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The following references are incorporated by reference herein:

U.S. Pat. No. 6,920,236
U.S. Pat. Pub. 20020075450
U.S. Pat. Pub. 2002/0136435
U.S. Pat. Pub. 2006/0087618
U.S. Pat. Pub. 2009/0021695
PCT Pat. Pub. WO2003/092482

1. A system for assessing diplopia, the system comprising:
a headset comprising:
a sensor configured to detect movement of the headset; and
a visual display configured to display a first object and a second object; and
a computer processor, wherein the computer processor is configured to:
receive an input from the sensor, wherein the input is correlated to movement of the headset from a first position to a second position;
transmit an output signal to move the first object or move the second object within the visual display, wherein the movement of the first or second object is in response to the first input received from the sensor; and
quantify the movement of the headset from the first position to the second position.

2. The system of claim 1 wherein:
the first object and the second object are not aligned in the visual display when viewed by a person with diplopia with the headset in the first position;
the first object and the second object are aligned in the visual display when viewed by the person with diplopia with the headset in the second position.

3. The system of claim 1 wherein the sensor is an accelerometer or magnetic sensor.

4. (canceled)

5. The system of claim 1 wherein the sensor is configured to detect rotational position data of the headset.

6. The system of claim 1 wherein the computer processor is configured to receive the input and transmit the output signal via a wireless transmission.

7. The system of claim 1 wherein the system is not configured to detect eye movement of a person when the person is wearing the headset.

8. The system of claim 1 further comprising an audio transmitter configured to provide audible instructions to a person during operation.

9. The system of claim 1 wherein the headset is a virtual reality headset.

10. The system of claim 1 wherein the visual display is configured to cover a field of view of a person wearing the headset.

11. A method of assessing diplopia in a person, the method comprising:
(i) displaying a first object and a second object in a visual display of a headset worn by the person;
(ii) detecting movement of the head of the person from a first position to a second position; and
(iii) moving the first object or the second object in the visual display of the headset in response to the movement of the head of the person, wherein:
the first object and the second object do not appear to the person to be aligned when the head of the person is in the first position; and
the first object and the second object do appear to the person to be aligned when the head of the person is in the second position.

12. The method of claim 11 wherein the movement is detected via a sensor coupled to the headset.

13. The method of claim 12 wherein the sensor is an accelerometer or magnetic sensor.

14. (canceled)

15. The method of claim 12 wherein the movement detected by the sensor is rotational movement.

16. (canceled)

17. The method of claim 12, further comprising transmitting data from the sensor to a computer processor, wherein the computer processor records movement of the head of the person from the first position to the second position.

18. The method of claim 17 wherein the movement of the head of the person from the first position to the second position is an indication of diplopia.

19. (canceled)

20. The method of claim 11 wherein the headset is a virtual reality headset.

21. The method of claim 11 further comprising providing instructions to the person to move the head of the person to align the first object and the second object, wherein providing instructions occurs after step (i) and before step (ii).

22. The method of claim 11 further comprising repeating steps (i), (ii) and (iii), wherein the first object and the second object are displayed in different locations in the visual display of the headset worn by the person in each iteration of step (i).

23. The method of claim 11 wherein the method does not comprise detecting eye movement of the person.

24. A method of assessing diplopia in a person, the method comprising:
displaying a first object and a second object in a visual display of a headset worn by the person, wherein:
the first object and the second object do not appear to the person to be aligned; and
the first object appears to the person to be moving along a first path having a first plurality of locations;
recording movement of the head of the person along a second path as the person attempts to align the first object with the second object, wherein the second path comprises a second plurality of locations;
comparing the first plurality of locations to the second plurality of locations to establish a plurality of deviation angles; and
recording the deviation angles of the movement of the head of the person along the second path.

25. The method of claim 24 wherein the first path of the first object extends across the visual field of the person.

26. The method of claim 24 wherein movement of the head of the person along the second path is recorded by a computer processor receiving an input signal from a sensor coupled to the headset.

27. The method of claim 26 wherein the sensor is an accelerometer or magnetic sensor.

28. (canceled)

29. The method of claim 26 wherein the sensor is configured to detect rotational position data of the headset.

30. (canceled)

31. The method of claim 24 wherein the method does not comprise detecting eye movement of the person.

32. The method of claim 24 further comprising providing audible instructions to the person during operation.

33. The method of claim 24 wherein the headset is a virtual reality headset.

34. The method of claim 24 the visual display is configured to cover a field of view of a person wearing the headset.
